(19) United States
(12) Patent Application Publication
(10) Pub. No.: US 2008/0153177 A1
(43) Pub. Date: Jun. 26, 2008

(54) TUMOUR MARKER IN BRCA2 ASSOCIATED BREAST CANCER

(76) Inventor: Hakan Olsson, Lund (SE)

Correspondence Address: GAUTHIER & CONNORS, LLP, 225 FRANKLIN STREET, SUITE 2300, BOSTON, MA, 02110, US

(21) Appl. No.: 11/953,228

(22) Filed: Dec. 10, 2007

Related U.S. Application Data
(63) Continuation of application No. PCT/SE2006/000678, filed on Jun. 9, 2006.

(30) Foreign Application Priority Data
Jun. 10, 2005 (SE) 0501329-7

(51) Int. Cl. G01N 33/53

(52) U.S. Cl. 436/548

(57) ABSTRACT

The present invention relates to a diagnostic method for diagnosing the possible recurrence of breast cancer, wherein the presence of the monoclonal antibody CA-125 is monitored in a BRCA2 tumor sample, whereby any presence of CA-125 is indicative of metastazing BRCA2. The invention also relates to the use of monoclonal antibody CA-125, or monoclonal antibody oregovo monoclonal antibody in the preparation of a therapeutic composition for the treatment of breast cancer of the BRCA2 type.

### TECHNICAL FIELD

The present invention relates to a tumour marker associated with breast cancer, which marker can be used for diagnosing presence of absence of breast cancer. In particular the marker relates to BRCA2 cancer.

### BACKGROUND OF THE INVENTION

CA-125 is a useful tumour marker for ovarian cancer (Hensley & Spriggs 2004). As such the marker is not specific for ovarian cancer. A number of other tumour diseases and conditions have been found to be related to elevated plasma levels of CA-125 (Sjövall et al 2002). In breast cancer overall 5-80% (median 30%) have been found to be positive for CA-125 in blood plasma. (Leonard et al 2004). However, in ovarian cancer it is still disputed whether the marker appears early enough during the course of the disease to be a good tool in screening for ovarian cancer (Hensley & Spriggs 2004). In general Its use has been best corroborated for metastatic disease and its disease course. The use of CA-125 has been incorporated Into the screening of ovarian cancer in hereditary settings such as patients with BRCA1 or BRCA2 germline mutations (Levavi & Sabah 2003). The value of the marker in ovarian screening of hereditary cases is disputed.

### SUMMARY OF THE INVENTION

In this invention it is shown that breast tumours in a subgroup of BRCA2 patients stains positive for CA-125 and patients have elevated plasma levels. Further that the elevations precludes the symptoms of recurrence or metastatic disease and seems to be an early disease marker that potentially could be used for therapeutic use.

In particular the invention relates to a method for diagnosing the possibility of breast cancer recurrence, wherein the presence of the monoclonal antibody CA-125 is monitored in BRCA2 tumours, whereby any presence of CA-125 is indicative of metastazing BRCA2.

In a preferred embodiment the invention concerns a method wherein the breast cancer comprises BRCA2 germline mutations.

In a further aspect of the invention it relates to the use of monoclonal antibody CA-125, or monoclonal antibody oregovomab in the preparation of a therapeutic composition for the treatment of recurrent breast cancer of the BRCA2 type.

In a preferred embodiment of this further aspect the invention relates to the use, wherein the breast cancer is BRCA2 germline mutations

### BACKGROUND

In patients having a strong hereditary predisposition for breast cancer (dominant inheritance) mutations in either BRCA1 or BRCA2 are responsible for ⅓ of the familiar heredited connection. BRCA1 mutations are twice as common as BRCA2 mutations in Sweden. About ½% of all breast cancer shows a BRCA2 mutation. In certain populations, as that of Island and in women of Azkenazy jewish origin there is a considerably higher ratio of breast cancer connected to BRCA2.

For the time being, it is uncertain if BRCA2 associated breast cancer shall be treated in a different way than other types of breast cancer. It has previously been found BRCA2 associated breast cancer follows a distinct genetic development when measured using CGH (Tirkonnen et al, 1999) or by means of its gene expression profile (Hedenfalk et al, 2001). Thus it is probable that tumour and blood will show different protein markers of the disease.

It has turned out, as shown below, that patients having a BRCA2 mutation and having breast cancer express in 6/14, (42% of women, 0% of men) the CA-125 in its breast tumour and in 2 of these, wherein the plasma levels have been studied, early-increased levels of CA-125 correlate to formation of malignant metastases before any presence of clinical symptoms.

Two out of 8 tumours of women having a sporadic breast cancer also express CA-125 In their breast tumour. The expression Is more focal and less than that found in BRCA2 patients. The question if sporadic breast cancer also shows increased levels of CA-125 in plasma is uncertain, but prior publications in the field shows that such a connection may exist in 5 to 80% (median 30%) but that the expressioni is shown at an advanced cancer when the clinical symptoms are already there.

CA-125 positivity at BRCA2 associated breast cancer is not correlated to any other histopathological factor, hormone receptor or age.

At ovarian cancer, which strongly expresses CA-125, there is an antibody denoted OvaRex (by Altarex) undergoing clinical testing.

It is postulated that the same antibody will have a positive therapeutical value in the treatment of CA-125 positive BRCA2 associated breast cancer. Further, the treatment results using conventional factors, such as chemotherapy and antihormonial therapy can be followed and the treatment can be controlled during any metastasis situation using CA-125 in patients using CA-125 expression in primary tumours.

At the diagnosis of BRCA2 associated tumour staining of CA-125 of the primary tumour will facilitate diagnosis and histogenesis.

### DETAILED DESCRIPTION OF THE PRESENT INVENTION

Material and Methods

Patients

The population studied comprised twenty-five patients, of which seventeen were BRCA2 germline mutation carriers. Eight patients, registered as having invasive breast carcinoma at the population based Regional Tumour Registry In Lund, Sweden, were randomly selected as control group. The median age was 53 years (range 27-86 years) in the BRCA2 group and 57 years (range 32-76 years) in the control group.

Histopathology

The twenty-five cases of BRCA2 tumours and sporadic tumours have been retrospectively classified. Surgical tumour specimens fixed in 10% buffered formalin solution were embedded in paraffin, routinely sectioned in 5μ sections and stained with haematoxylin-eosin. All available pathological tissue was re-examined by one pathologist without knowing whether the sections belonged to the hereditary or the population-based comparison group. The following parameters were considered: non-invasive tumour; invasive tumour; histological grade (tubules, nuclear pleomorphism, mitotic count); % tumour as solid sheets; pushing margins; necrosis; vascular invasion and lymphoplasmacytic infiltrates.

Immunohistochemistry

Paraffin section (5μ thick) were cut and mounted on silane glass slides. Deparaffinized sections were processed for the immunohistochemical demonstration of the monoclonal antibody, anti-CA-125 (clone M11, code no M3520, DAKOCytomatlon, Copenhagen, Denmark, working dilution 1:100) using Chem-mate kit (DAKOCytomation, Copenhagen, Denmark).

Serum Levels of CA-125

The serum level of CA-125 was determined with the standard method employed at the University Hospital, Lund. Reference normal value <10 kU/L.

Mutation Testing

The method of mutation screening for BRCA has previously been described (Loman et al 1998). A combination of heteroduplex testing by DHPLC, protein truncation tests and direct sequencing has been employed to detect mutations causing the disease. The laboratory used performs BRCA1 and BRCA2 testing for the majority of hereditary breast and ovarian families in Sweden.

Results

TABLE 1
CA-125 positivity in relation to histopathological parameters in BRCA2 cases and controls.

| | | age at | | non- | | histological | | | | CA-125 |
|---|---|---|---|---|---|---|---|---|---|---|
| Patient no | sex | diagnosis | | invasive | invasive | grade | ER | PGR | pTNM | immunoreactivity |
| 1 | F | 40 | BRCA2 | — | Ductal | 3 | − | − | pT2N1M0 | − |
| 2 | F | 54 | BRCA2 | DCIS | Ductal | 3 | + | (+) | pT1cN2M0 | − |
| 3 | F | 44 | BRCA2 | DCIS | Ductal | 3 | − | − | pT1cN0M0 | + |
| 4 | F | 34 | BRCA2 | DCIS | Ductal | 3 | + | + | pT1cN0M0 | − |
| 5 | F | 27 | BRCA2 | DCIS | Ductal | 3 | (+) | + | pT2N1M0 | + |
| 6 | F | 83 | BRCA2 | DCIS | Ductal | 3 | + | − | pT2N2M0 | − |
| 7 | F | 51 | BRCA2 | DCIS | Ductal | 2 | + | − | pT1cN0M0 | − |
| 8 | F | 58 | BRCA2 | DCIS | Ductal | 2 | + | + | pT2N1aM0 | − |
| 9 | F | 28 | BRCA2 | DCIS | Ductal | 3 | (+) | (+) | pT2N2M0 | − |
| 10 | F | 61 | BRCA2 | DCIS | Ductal | 3 | − | (+) | pT1cN1aM0 | + |
| 11 | F | 49 | BRCA2 | LCIS | Lobular | 2 | + | + | pT3N3aM0 | + |
| 12 | F | 63 | BRCA2 | DCIS | Ductal | 2 | + | − | pT2N3aM0 | − |
| 13 | F | 38 | BRCA2 | LCIS | Lobular | 2 | | | pT2NXM0 | − |
| 13 | F | 39 | BRCA2 | — | Lobular | 3 | + | + | pT2N0M0 | + |
| 13 | F | 44 | BRCA2 | — | Metastases | — | + | + | | + |
| 14 | F | 46 | BRCA2 | | Ductal | | − | − | | Block not found* |
| 14 | F | 57 | BRCA2 | DCIS | Ductal | 2 | | | pT1bN0M0 | + |
| 14 | F | 65 | BRCA2 | — | Metastases | — | + | + | | − |
| 15 | M | 86 | BRCA2 | — | Papillar | 3 | + | + | pT1cNXM0 | − |
| 16 | M | 77 | BRCA2 | — | Ductal | 3 | + | + | pT4bN1M0 | − |
| 17 | M | 68 | BRCA2 | — | Ductal | 3 | + | + | pT2N0M0 | − |
| 18 | F | 60 | sporadic | DCIS | Ductal | 3 | + | − | pT4bN0M0 | + |
| 19 | F | 49 | sporadic | DCIS | Ductal | 2 | + | + | pT2N1bM0 | − |
| 20 | F | 54 | sporadic | — | Ductal | 1 | + | + | pT1cN0M0 | − |
| 21 | F | 62 | sporadic | — | Ductal | 2 | − | − | pT1cN1aM0 | − |
| 22 | F | 76 | sporadic | — | Ductal | 1 | − | + | pT1cN1bM0 | − |
| 23 | F | 67 | sporadic | DCIS | Ductal | 1 | + | + | pT1cNXM0 | − |
| 24 | F | 57 | sporadic | DCIS | Ductal | 2 | + | − | pT1cN0M0 | − |
| 25 | F | 32 | sporadic | — | Ductal | 3 | − | − | pT3NXM0 | + |

ER = estrogen receptor,
PGR = progesterone receptor,
+ = positive,
(+) = weakly positive,
− = negative.
LCIS = lobular cancer in situ,
DCIS = ductal cancer in situ.

In FIG. 1 it is shown CA-125 positivity in a patient with recurrent disease in relation to therapy. The patient presented with bilateral breast cancer 98199 (both stage 1 of lobular type). After surgery she was given 7 cycles of CMF and radiotherapy was administered to the right breast. The serum level of CA-125 was normal in November 2000. In April 2001 (arrow 1) the level started to rise (62 kU/L), due to a further rise in May (82 kU/L), June (120 kU/L) and July (208 kU/L) a diagnostic salpingo-oophorectomy was done in the patient, who had no prior symptoms. At the operation (arrow 2) bilateral breast cancer met stases were found in the ovaries. The recurrence was hormone receptor positive and histologically clearly of same histology as one of the previous breast cancers. No further metastases were detected. The patient was started on tamoxifen. In September 2002 CA-125 levels were rising (420 kU/L) again in the patient, without having had any symptoms of further metastases. The levels continued to rise.

December 2002 (676 kU/L) with the patient being symptomless, a skeletal investigation was performed (scintigram, X-ray) demonstrating multiple metastases in the lumbar spine (arrow 3). Tamoxifen was then changed to therapy with an aromatase inhibitor, anastrazole, and a biphosphonate. Levels of CA-125 improved, April 2003 (76 kU/L) and in August 2003 (145 kU/L). In the autumn 2003 serum CA-125 was again rising September 2003 (209 kU/L, October 2003 (447 kU/L), November 2003 (599 kU/L), December 2003 (931 kU/L) and in February 2004 (1877 kU/L). In March 2004, when CA-125 levels reached 2393 kU/L, the patient's haemoglobin levels started to decline and after a further increase of CA-125 (4273 kU/L) in June a bone marrow biopsy showed that the marrow was heavily infiltrated with cancer cells. The endocrine therapy was discontinued and therapy with FEC was started (arrow 4).

A rapid decline of CA-125 levels has then been seen with Improvement of haemoglobin levels and no other symptoms. Levels in December of 2004 are now 150 kU/L. A retrospective investigation of the breast tumours 98/99 has found that one of the tumours show a heavy positivity for CA-125 while the other tumour is negative for Immunohistochemical staining (patient 13, see table 1 and FIG. 3).

FIG. 2 shows the CA-125 positivity in a patient with recurrent disease in relation to therapy. Patient number 2 presented in December 2002 (at age 65) with metastases to the pleura and lung after having bilateral breast cancer at age 46 years of age (stage I, right breast) and at 57 years of age (stage I, left breast). Follow up between age 57 and 65 had not proven any tumour recurrence. CA-125 started to rise already in February 2002 and as significantly elevated in October 2002. Gynaecological examination did not reveal any varian tumour and two months later symptoms appeared from lung/pleura where metastases were shown from breast cancer (arrow). Because of hormone receptor positivity therapy with aromatase inhibitor was initiated. The patients initial breast tumour at age 57 stained positive for CA-125 while blocks for the breast tumour at age 46 were not retrievable (see table 1, patient number 14).

The results of this study indicate that CA-125 is a valuable breast tumour marker for a subset of BRCA2 patients. Approximately ⅓ ( 6/14 females) of the patients had tumours staining positive for CA-125. Further in two patients, the only with metastatic disease, the serum elevation of CA-125 preceded the onset of symptoms with months suggesting that the marker is a clinical useful early marker. In breast cancer in general, elevated serum CA-125 has been suggested to be a marker of advanced disease (Norum et al 2001) and its serum levels has not been considered to be a valuable tumour marker (Ogmundsdottir et al 1996).

In cases with ovarian carcinoma, where the marker so far has found usage clinically, its value for guiding therapy or detecting primary or metastatic disease is still controversial although a number of studies have pointed to its possible value (Hensley & Spriggs 2004, Cannistra 2004). A murine monoclonal antibody for therapy, OvaRex Altarex (oregovomab), has recently been developed and phase I-III studies are on going in ovarian cancer (Berek 2004). Initial experience is promising both regarding therapeutic effects and side effects. Present data of the present invention suggest that the therapeutic antibody is of value also for a subset of BRCA2 patients.

In the patient series of BRCA2 cases the presence of CA-125 positivity in tumour cells were not significantly associated with other known tumour parameters such as histological grade (tubules, nuclear pleomorphism, mitotic count); % tumour as solid sheets; pushing margins; necrosis; vascular invasion and lymphoplasmacytic infiltrates. Further positivity was not related to the patient itself because one patient with bilateral tumours displayed discordance in CA-125 positivity between the two breast tumours. In the BRCA2 series 3 men with breast cancer were included all of whom did not express CA-125 In their breast tumours.

Of the sporadic cases (8 untested female cases without a family history) 2 expressed CA-125 immunoreactivity. One of these patients was very young (aged 30 at diagnosis) and it has not been definitively excluded that she may be a gene carrier. Neither hormone receptor positivity nor histological type correlated with CA-125 positivity.

In a previous article we have hypothesised that the development of BRCA2 tumours is related to a breast tissue that is more developed/differentiated than cases with BRCA1 tumours (Olsson H. Tumour biology of a breast cancer at least partly reflects the biology of the tissuelepithelial cell of origin at the time of initiation—a hypothesis. The Journal of Steroid Biochemistry & Molecular Biology 2000;74:345-50) in line with the finding that BRCA2 tumours both display ER+ and ER− tumours while BRCA1 tumours mainly are ER− (Loman et a 1998). The development of BRCA2 tumours therefore could start in an intermediate cell between the basal type (suggested to be the origin for BRCA1) and the hormone receptor positive luminal type (Perou et al 1999, 2000, Sörlie et al 2003). Thus it is therefore postulated that at least at subset of these intermediate cells has a high CA-125 content. It is from our data and literature also clear that a subset of sporadic breast cancer also express CA-125 in the serum (Leonard et al 2004). Available data suggest however that this is a late event in the dissemination of the tumour disease.

The findings here will also prompt physician not only to suspect ovarian cancer in relation to a CA-125 elevation but also consider the possibility that the BRCA2 mutation carrier with elevated plasma levels has a primary or metastatic breast cancer and that in this patient group clinical trials assessing the role of the commercial available antibody OvaRex Mab in early and late disease should be given high priority. In sporadic cases further studies are needed to assess the potential diagnostic and therapeutic role of CA-125.

### FIGURE LEGENDS

FIG. 1. CA-125 positivity is shown in a patient with recurrent disease in relation to therapy. The patient presented with bilateral breast cancer 98/99 (both stage I of lobular type). After surgery she was given 7 cycles of CMF and radiotherapy was administered to the right breast. The serum level of CA-125 was normal in November 2000. In April 2001 (arrow 1) the level started to rise (62 kU/L), because of further rise in May (82 kU/L), June (120 kU/L) and July (208 kU/L) a diagnostic salpingo-oophorectomy was done in the patient who had no prior symptoms. At the operation (arrow 2) bilateral breast cancer metastases were found in the ovaries. The recurrence was hormone receptor positive and histologically clearly of same histology as one of the previous breast cancers. No further metastases were detected. The patient was started on tamoxifen. In September 2002 CA-125 levels were rising (420 kU/L) without that the patient had any symptoms of further metastases. The levels continued to rise.

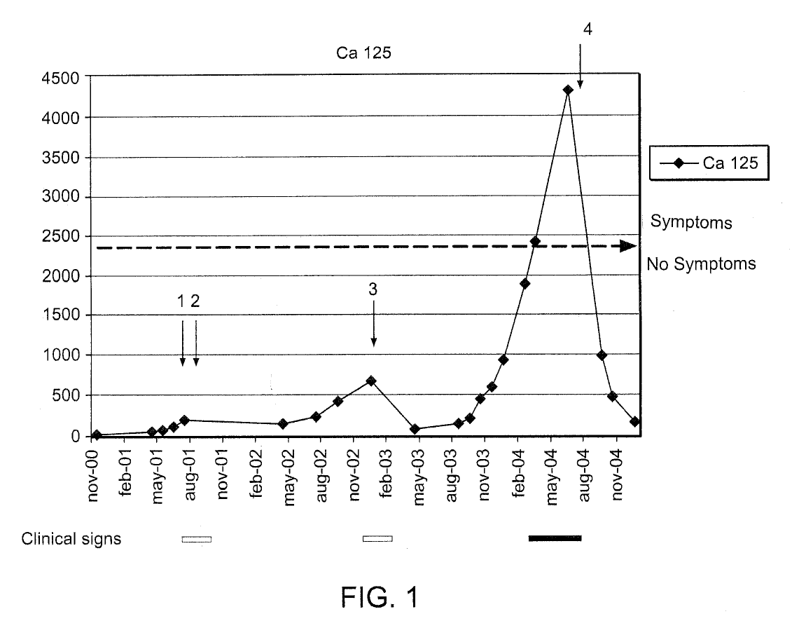

FIG. 2. CA-125 positivity is shown in a patient with recurrent disease in relation to therapy. Patient number 2 presented in December 2002 (at age 65) with metastases to the pleura and lung after having bilateral breast cancer at age 46 years of age (stage I, right breast) and at 57 years of age (stage I, left breast). Follow up between age 57 and 65 had not proven any tumour recurrence. CA-125 started to rise already In February 2002 and was significantly elevated in October 2002. Gynaecological examination did not reveal any ovarian tumour and two months later symptoms appeared from lung/pleura where metastases were shown from breast cancer (arrow). Because of hormone receptor positivity therapy with aromatase inhibitor was initiated. The patients initial breast tumour at age 57 stained positive for CA-125 while blocks for the breast tumour at age 46 were not retrievable (see table 1, patient number 14).

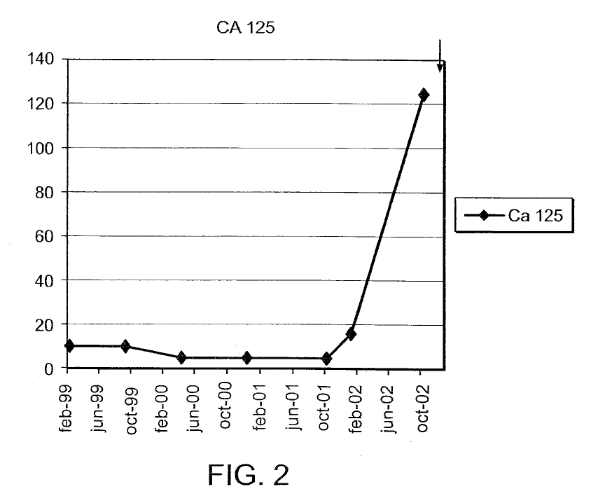

FIG. 3. Examples of CA-125 immunoreactivity in BRCA2 associated breast cancer are shown. The staining shows a strong membrane and cytoplasmatic staining of tumour cells in patient number 3(a) and in patient number 10(b). In one of the patients (number 13) with metastatic disease you can se a strong immunoreactivity in both the primary tumour (c) and in the metastases in the bone marrow (d).

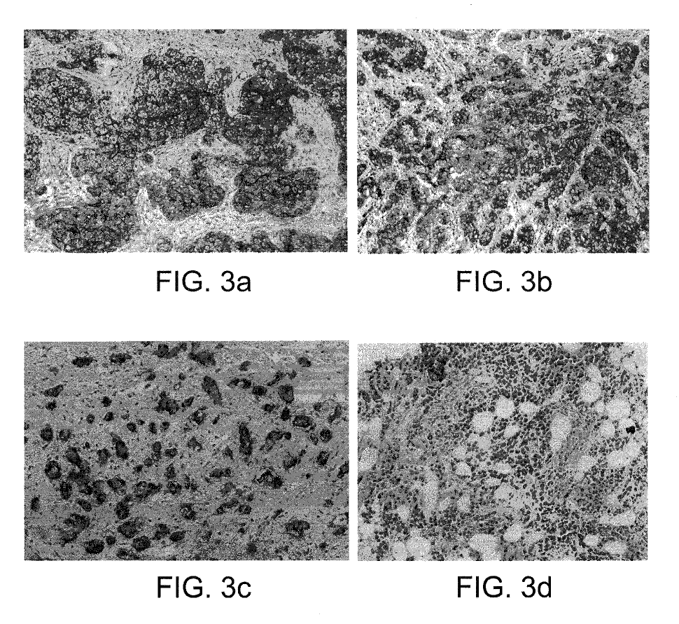

FIG. 4. Examples of CA-125 immunoreactivity in sporadic breast cancer are shown. Membrane and cytoplasmatic immunoreactivity in the tumour from patient number 18(a) and focally distributed immunoreactivity in the tumour from patient number 25(b).

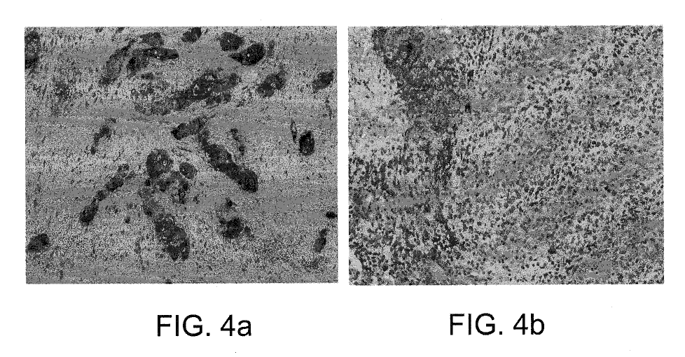

### REFERENCES

Hensley M L & Spriggs D R. Cancer screening: How good is good enough? Journal of Clinical Oncology 2004; 22: 20: 4037-4039.

Leonard G D, Low J, Berman A W, Swain S M, CA-125 elevation in breast cancer: A case report and review of the literature.

The Breast Journal 2004; 10: 2: 146-149.

Sjövall K, Nilsson B, Einhorn N. The significance of serum CA-125 elevation in malignant and non-malignant diseases.

Gynecological Oncology 2002; 85: 175-178.

Norum L F, Erikstein B, Nustad K. Elevated CA-125 in breast cancer—A sign of advanced disease.

Tumour Biol 2001; 22(4): 223-228.

Ogmundsdottir H M, Gudlaugsdottir S, Bjornsson J, Jonasdottir S. Altered expression of CA-125 in breast carcinomas.

APMIS 1996; 104(1): 47-53.

Bjorge T, Lie A K, Hovig E et al. BRCA1 mutations in ovarian cancer and borderline tumours in Norway: a nested case control study.

BrJ Cancer 2004; 15; 91: 1829-1834.

Cannistra S A. Cancer of the ovary.

N Engl J Med 2004; 351:2519-2529.

Levavi H, Sabah G. BRCA susceptibility genes-a review of current conservative management of mutation carriers.

Eur J Gynaecol Oncol 2003; 24: 463-6.

Loman N, Johannsson O, Bendahl P-O, Fernö M, Olsson H, Borg Å. Steroid receptors in hereditary breast cancer related to BRCA1 and BRCA2 or unknown susceptibility genes. Cancer 1998;83:310-9.

Olsson H. Tumour biology of a breast cancer at least partly reflects the biology of the tissue/epithelial cell of origin at the time of initiation—a hypothesis.

The Journal of Steroid Biochemistry & Molecular Biology 2000;74:345-50.

Perou, C. M. , Jeffrey, S. S., van de Rijn, M. , et al. (1999) Distinctive gene expression patterns in human mammary epithelial cells and breast cancers.

Proc. Natl. Acad. Sci. USA 96, 9212-9217.

Perou C M, Sorlie T, Eisen M B et al. Molecular portraits of human breast tumours.

Nature 2000; 406; 747-752.

Sorlie T, Tibshirani R, Parker J, et al. Repeated observation of breast tumor subtypes in independent gene expression data sets.

Proc Natl Aced Sci U S A. Jul. 8, 2003;100(14):8418-23.

Berek J S. Immunotherapy of ovarian cancer with antibodies: focus on oregovomab.

Expert Opin Biol Ther. July 2004;4(7):1159-65

1. A method for diagnosing the possibility of early breast cancer recurrence, wherein the presence of monoclonal antibody CA-125 is monitored in BRCA2 tumours is determined in isolated blood plasma whereby any presence of CA-125 is indicative of metastazing BRCA2.

2. The method according to claim 1, wherein the breast cancer comprises BRCA2 germline mutations.

3. A use of monoclonal antibody CA-125 or monoclonal antibody oregovo monoclonal antibody in the preparation of a therapeutic composition for the treatment of recurrent breast cancer of the BRCA2 type or primary tumour of breast cancer of the BRCA2 type.

4. The use according to claim 3, wherein the breast cancer is BRCA2 germline mutations.
